(19) United States
(12) Patent Application Publication
(10) Pub. No.: US 2012/0308491 A1
(43) Pub. Date: Dec. 6, 2012

(54) DENTIFRICE COMPOSITIONS

(75) Inventors: Ramachandra Shastry, Dayton, NJ (US); James Masters, Ringoes, NJ (US)

(73) Assignee: COLGATE-PALMOLIVE COMPANY, New York, NY (US)

(21) Appl. No.: 13/521,320

(22) PCT Filed: Jan. 14, 2011
(86) PCT No.: PCT/US11/21385

§ 371 Date: Jul. 10, 2012

Related U.S. Application Data
(60) Provisional application No. 61/294,851, filed on Jan. 14, 2010.

(51) Int. Cl. A61K 8/64; A61Q 11/00

(52) U.S. Cl. 424/50

(57) ABSTRACT

Described herein are dentifrice compositions comprising a zinc-containing salt and carboxypeptidase, wherein the zinc-containing salt is not sequestered.

### CROSS REFERENCE TO RELATED APPLICATIONS

This application claims priority to U.S. Provisional Patent Application No. 61/294,851, filed on 14 Jan. 2010, which is incorporated herein by reference.

### BACKGROUND

Dental plaque is present to some degree, in the form of a film, on virtually all dental surfaces. It is a byproduct of microbial growth, and comprises a dense microbial layer consisting of a mass of microorganisms embedded in a polysaccharide matrix. Plaque adheres firmly to dental surfaces and is removed only with difficulty even through a rigorous brushing regimen. Moreover, plaque rapidly reforms on the tooth surface after it is removed. The danger associated with the formation of plaque on the teeth lies in the tendency of plaque to build up and eventually produce gingivitis, periodontitis and other types of periodontal disease, as well as dental caries and dental calculus.

Conventional zinc-containing oral formulations have been largely unsuccessful because of the inability of the zinc ions, contained therein, to penetrate the plaque matrix to a sufficient extent. As a result, conventional zinc-containing formulations demonstrate limited efficacy against plaque. Conventional zinc-containing oral formulations also provide an undesirable astringent taste, especially when zinc is present at higher concentrations.

Accordingly, there exists a need for zinc-containing formulations that provide increased penetration of zinc ions in the plaque matrix, and increased anti-plaque efficacy.

### SUMMARY

The above goals may be achieved by incorporating a carboxypeptidase with zinc in an oral formulation, whereby a controlled-release effect is achieved. In accordance with the present invention, it has been determined that the use of carboxypeptidase and a zinc-containing compound in oral compositions provides enhanced anti-plaque and fresh breath effects over a prolonged period of time, while reducing astringent taste. The effects of the zinc and the carboxypeptidase are each synergistically improved by the presence of the other in the formulation and the amount of zinc necessary to achieve the desired effects of the formulation is reduced.

### DETAILED DESCRIPTION

As used herein, “dentifrice” refers to a paste, gel, lozenge, gum, or liquid formulation, and excludes hardenable compositions used in the mouth. In some embodiments, the dentifrice is deep striped, surface striped, or multilayered.

The expressions “carrier” or “aqueous carrier” as used throughout this description denote any safe and effective materials for use herein. Such materials include, for example, thickening agents, humectants, ionic active ingredients, buffering agents, anticalculus agents, abrasive polishing materials, peroxide sources, alkali metal bicarbonate salts, surfactants, titanium dioxide, coloring agents, flavor systems, sweetening agents, antimicrobial agents, herbal agents, desensitizing agents, stain reducing agents, and mixtures thereof.

As used herein, the term “sequester” or “sequestered” refers to the encapsulation, isolation, segregation, etc. of one or more components or ingredients, from the remainder of the components or ingredients in a particular formulation.

Some embodiments of the present invention provide a dentifrice composition comprising a zinc-containing salt and a carboxypeptidase, wherein said zinc-containing salt is not sequestered. In some embodiments, the penetration of zinc into the plaque matrix is increased. In some embodiments, the carboxypeptidase forms a labile complex with zinc. In some embodiments, the carboxypeptidase-zinc complex breaks down proteins in the plaque matrix. Some embodiments provide a controlled-release of the zinc-containing salt.

In some embodiments, the combination of carboxypeptidase and the zinc-containing salt provides an enhanced effect because the zinc-enzyme complex remains active upon entry into the plaque matrix. The activity of the carboxypeptidase as well as the increased concentration of zinc within the plaque matrix is believed to increase the antibacterial, anti-plaque, and anti-malodor effects of the formulation.

The unique binding mechanism of zinc to carboxypeptidase in this formulation results in a loose association between the two. The association constant between zinc and carboxypeptidase in the formulation allows zinc to dissociate from the complex after uptake into the plaque matrix, leading to a controlled-release effect and a longer duration of action. In this manner, the zinc does not have the effect of inactivating the enzyme and each component is able to work with enhanced efficacy. In some embodiments, less of each component can be used in the formulation because the independent effects of the zinc and the carboxypeptidase are amplified when they are applied in combination with each other.

Some embodiments of the present invention provide enhanced uptake of zinc into the plaque matrix; and thus, the total quantity of zinc used in the formulation may be reduced. In this way, the astringent taste associated with the presence of zinc is diminished as well. The astringency of the formulation is further reduced due to the complexation of the astringent ion to carboxypeptidase.

The zinc compounds that provide zinc ions for use in combination with carboxypeptidase may be any physiologically acceptable zinc salt including the water soluble (including sparingly water soluble) organic and inorganic zinc salts which provide at least about 0.01 mg of zinc ions per ml of water. The water-soluble zinc salts (at least 1% soluble) are preferred, especially the zinc halides and zinc acetate. More preferred are sparingly soluble zinc salts, of which zinc citrate, zinc chloride, or zinc nitrate are most preferred. Examples of suitable zinc salts that may be employed include: zinc acetate, zinc fluoride, zinc ammonium sulfate, zinc formate, zinc bromide, zinc iodide, zinc chloride, zinc nitrate, zinc chromate, zinc phenol sulfonate, zinc citrate, zinc salicylate, zinc dithionate, zinc sulfate, zinc fluosilicate, zinc gluconate, zinc tartarate, zinc succinate, zinc glycerophosphate, and mixtures thereof. Other suitable zinc salts are disclosed in U.S. Pat. No. 4,138,477 having a solubility of at least about 0.01 mg of zinc ions per ml of water, the disclosure of which is incorporated herein by reference in its entirety.

The zinc salt can be present in amounts that provide about 0.01-5% by weight of zinc ions and preferably about 0.02-1% of zinc ions by weight in the oral composition. Most preferably, the zinc is present in an amount that provides about 0.3-0.6% by weight of zinc ions. Depending on the formulation used, and the amount of carboxypeptidase, a person having ordinary skill in the art will be capable of determining the amount of zinc to incorporate into the composition to provide the desired amount of zinc ions. Preferably, the amount of zinc used in the dentifrice composition of the preferred embodiments is within the range of from about 0.01% to about 2% by weight.

The carboxypeptidase preferably is present in amounts that provide about 0.01-5% by weight of carboxypeptidase in the formulation. Preferably, the carboxypeptidase is present at about 0.1-1% by weight, and most preferably the carboxypeptidase is present at about 0.5% by weight.

The ratio of zinc to carboxypeptidase in the formulation of the preferred embodiments can range from about 5:1 to 1:5. Preferably, the ratio of zinc to carboxypeptidase is about 3:1 to 1:3, and more preferably the ratio is about 2:1 to 1:2. Most preferred is a 1:1 ratio of zinc to carboxypeptidase. The composition of the invention can be incorporated into various dentifrice formulations including toothpastes, mouthwashes, tooth powders, and the like.

### Dentifrice Vehicle

Orally-acceptable vehicles used to prepare the dentifrice component of the present invention may include a water-phase containing a humectant. The humectant is preferably glycerin, sorbitol, xylitol, and/or propylene glycol of molecular weight in the range of 200 to 1,000; but, other humectants and mixtures thereof may also be employed. The humectant concentration typically totals about 5 to about 70% by weight of the oral composition.

Reference hereto to sorbitol refers to the material typically commercially available as a 70% aqueous solution. Water is present typically in amount of at least about 10% by weight, and generally about 25 to 70% by weight of the dentifrice component. Water employed in the preparation of commercially suitable oral compositions should preferably be deionized and free of organic impurities. These amounts of water include the free water which is added plus that which is introduced with other materials such as with sorbitol.

### Abrasives

Abrasives that may be used ion preparing the dentifrice compositions include silica abrasives such as precipitated silicas having a mean particle size of up to about 20 microns, such as Zeodent 115, marketed by J.M. Huber Chemicals Division, Havre de Grace, Md. 21078, or Sylodent 783 marketed by Davison Chemical Division of W.R. Grace & Company. Other useful dentifrice abrasives include sodium metaphosphate, potassium metaphosphate, tricalcium phosphate, dihydrated dicalcium phosphate, aluminum silicate, calcined alumina, bentonite or other siliceous materials, or combinations thereof.

Preferred abrasive materials useful in the practice of the preparation of the dentifrice components in accordance with the present invention include silica gels and precipitated amorphous silica having an oil absorption value of less than 100 cc/100 g silica and preferably in the range of from about 45 cc/100 g to less than about 70 cc/100 g silica. These silicas are colloidal particles having an average particle size ranging from about 3 microns to about 12 microns, and more preferably between about 5 to about 10 microns and a pH range from 4 to 10 preferably 6 to 9 when measured as a 5% by weight slurry.

Oil absorption values are measured using the ASTM Rub-Out Method D281. The low oil absorption silica abrasive is present in the dentifrice compositions of the present invention at a concentration of about 5 to about 40% by weight and preferably about 10 to about 30% by weight.

Low oil absorption silica abrasives particularly useful in the practice of the present invention are marketed under the trade designation Sylodent XWA by Davison Chemical Division of W.R. Grace & Co., Baltimore, Md. 21203. Sylodent 650 XWA. This silica abrasive is a silica hydrogel composed of particles of colloidal silica having a water content of 29% by weight averaging from about 7 to about 10 microns in diameter and an oil absorption of less than 70 cc/100 g of silica and is a preferred example of a low oil absorption silica abrasive useful in the practice of the present invention.

The dentifrice composition of the present invention can contain a variety of optional dentifrice ingredients. As described below, such optional ingredients can include, but are not limited to, thickening agents, surfactants, antitartar agents, a source of fluoride ions, stabilizers, a synthetic anionic polycarboxylate, a flavoring agent, and coloring agents.

### Thickening Agents

Thickeners suitable of use in the composition of the present invention include natural and synthetic gums and colloids. Suitable thickeners include naturally occurring polymers such as carrageenans, xanthan gum, polyglycols of varying molecular weights sold under the tradename Polyox, and polyvinylpyrrolidone. Compatible inorganic thickeners include amorphous silica compounds which function as thickening agents and include colloidal silicas compounds available under the trade designation Cab-o-sil manufactured by Cabot Corporation and distributed by Lenape Chemical, Bound Brook, N.J.; Zeodent 165 from J. M. Huber Chemicals Division, Havre de Grace, Md. 21078; and Sylodent 15, available from Davison Chemical Division of W. R. Grace Corporation, Baltimore, Md. 21203. Other inorganic thickeners include natural and synthetic clays such as hectorite clays, lithium magnesium silicate (laponite) and magnesium aluminum silicate (Veegum).

The thickening agent preferably is present in the dentifrice composition in amounts of about 0.1 to about 10% by weight, preferably about 0.5 to about 4.0% by weight.

### Surfactants

Surfactants may be used in the composition of the present invention to achieve increased prophylactic action and render the dentifrice compositions more cosmetically acceptable. The surfactant preferably is a detersive material that imparts to the composition detersive and foaming properties.

Examples of enzyme compatible surfactants include nonanionic polyoxyethylene surfactants such as Pluronic F127, Polyoxamer 407, Steareth 30, Polysorbate 20, and amphoteric surfactants such as cocamidopropyl betaine and cocamidopropyl betaine lauryl glucoside. Preferred surfactants include a combination of pluronic F 127, Polyoxamer 407, Polysorbate 20, and cocamidopropyl betaine at a total surfactant concentration in the dentifrice composition of between about 2 to about 10% by weight and preferably between about 3.5 to about 6.5% by weight at weight ratios of 2.5 Polyaxomer 407, 2.5 PEG-40 castor oil, 3.3 Polysorbate-20 and 1.0 cocamidopropyl betaine.

### Fluoride and Other Active Agents

The dentifrice composition of the present invention may also contain a source of fluoride ions or fluorine-providing component, as anticaries agent in amount sufficient to supply about 25 ppm to 5,000 ppm of fluoride ions and include inorganic fluoride salts, such as soluble alkali metal salts. For example, preferred fluoride sources which are compatible with enzymes present in the composition are sodium fluoride, potassium fluoride, sodium fluorosilicate, ammonium fluorosilicate, as well as tin fluorides, such as stannous fluoride and stannous chloride. Sodium fluoride is preferred.

In addition to fluoride compounds, there may also be included antitartar agents such as pyrophosphate salts including dialkali or tetraalkali metal pyrophosphate salts such as Na₄P₂O₇, K₄P₂O₇, Na₂K₂P₂O₇, Na₂H₂P₂O₇ and K₂H₂P₂O₇ sodium tripolyphosphate, long chain polyphosphates such as sodium hexametaphosphate and cyclic phosphates such as sodium trimetaphosphate. These antitartar agents are included in the dentifrice composition at a concentration of about 1 to about 5% by weight.

### Enzyme Stabilizing Agents

The dentifrice composition of the present invention may also contain ingredients that stabilize enzymes in a dentifrice environment. These stabilizers protect the enzyme from inactivation by chelating metal impurities present in the dentifrice composition. Chelating agents include, ethylene diamine tetraacetic acid (EDTA) and sodium gluconate at concentrations between 0.01 and 1%, preferably between 0.1 and 0.5%. Other stabilizers may also prevent oxidation of amino acids, such as cysteine, that are critical for enzyme activity. Examples of agents that stabilize the enzyme against oxidation include sodium bisulfite, metal gallates, sodium stannate and ascorbic acid at concentrations between about 0.1 and about 1.5%, preferably between about 0.3 and about 0.75%.

### Anionic Polycarboxylate

Synthetic anionic polycarboxylates may also be used in the dentifrice compositions of the present invention as an efficacy enhancing agent for any antibacterial, antitartar or other active agent within the dentifrice composition. Such anionic polycarboxylates are generally employed in the form of their free acids or preferably partially or more preferably fully neutralized water-soluble alkali metal (e.g. potassium and preferably sodium) or ammonium salts. Preferred are 1:4 to 4:1 copolymers of maleic anhydride or acid with another polymerizable ethylenically unsaturated monomer, preferably methylvinylether/maleic anhydride having a molecular weight (M.W.) of about 30,000 to about 1,800,000 most preferably about 30,000 to about 700,000. Examples of these copolymers are available from GAF Corporation under the tradename Gantrez®, e.g. AN 139 (M.W. 500,000), AN 119 (M.W. 250,000); S-97 Pharmaceutical Grade (M.W. 700,000), AN 169 (M.W. 1,200,000-1,800,000), and AN 179 (M.W. above 1,800,000); wherein the preferred copolymer is S-97 Pharmaceutical Grade (M.W. 700,000).

When present, anionic polycarboxylates can be employed in amounts effective to achieve the desired enhancement of the efficacy of any antibacterial, antitartar or other active agent within the dentifrice composition. Generally, the anionic polycarboxylates are present within the dentifrice composition from about 0.05% to about 4% by weight, preferably from about 0.5% to about 2.5% by weight.

### Flavor

The dentifrice composition of the present invention may also contain a flavoring agent. Flavoring agents that are used in the practice of the present invention include essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Of these, the most commonly employed are the oils of peppermint and spearmint.

The flavoring agent is incorporated in the dentifrice composition at a concentration of about 0.1 to about 5% by weight and preferably about 0.5 to about 1.5% by weight.

### Other Ingredients

Various other materials may be incorporated in the dentifrice compositions of this invention, including desensitizers, such as potassium nitrate; whitening agents; preservatives; silicones; coloring agents; and chlorophyll compounds. These additives, when present, are incorporated in the dentifrice composition in amounts that do not substantially adversely affect the properties and characteristics desired.

Antibacterial agents may be incorporated in the dentifrice compositions of the invention. Common antibacterial agents used in oral care include triclosan, chlorohexidine, cetyl pyridinium chloride, and other quaternary amines. These agents, when present, are incorporated in the dentifrice composition in effective amounts that do not substantially adversely affect the desired properties and characteristics of the composition.

### Preparation of Dentifrice Compositions

To prepare a dentifrice composition of the present invention, the zinc and carboxypeptidase are preferably dissolved in water before adding other ingredients. Generally the humectants such as glycerin, sorbitol are dispersed in the water in a conventional mixer under agitation. Into the dispersion are added organic thickeners, such as carboxymethyl cellulose; antitartar agents such as tetrasodium pyrophosphate, sodium tripolyphosphate and any sweeteners. The resultant mixture is agitated until a homogeneous gel phase is formed. Into the gel phase are added a pigment such as TiO2, and any acid or base required to adjust the pH in the range of 6.4 to 7.3. These ingredients are mixed until a homogenous phase is obtained. Thereafter a premix of cetyl pyridinium chloride, enzyme and a reducing agent such as potassium stannate in an aqueous humectant solution is added and admixed with the homogeneous gel phase The resultant mixture is then transferred to a high speed/vacuum mixer; wherein, the thickener, and surfactant ingredients are added to the mixture. Thereafter the abrasive is added. Any water insoluble antibacterial agent, such as Triclosan, is solubilized in the flavor oils to be included in the composition and the solution is added along with the surfactants to the mixture, which is then mixed at high speed for from 5 to 30 minutes, under vacuum of from about 20 to 50 mm of Hg, preferably about 30 mm Hg. The resultant product is in each case a homogeneous, semi-solid, extrudable paste or gel product.

### Preparation of Liquid Oral Compositions

In the aspect of the present invention wherein the oral composition is substantially liquid in character such as a mouthwash or rinse, the vehicle is typically a water, humectant, alcohol mixture. The alcohol is a non-toxic alcohol such as ethanol or isopropanol. A humectant such as glycerine, sorbitol or an alkylene glycol such as polyethylene glycol or propylene glycol may be present in an amount of about 10 to 30% by weight, the oral rinse containing greater than about 45% by weight water and preferably about 50 to 85% by weight water, about 0 to 20% by weight of a non-toxic alcohol and about 10 to 40% by weight of the humectant. A thickener such as a Pluronic may be present at a concentration of about 1.0 to about 3.0% by weight, cetyl pyridinium chloride at a concentration of about 0.02 to about 1.0% by weight, a reducing agent such potassium stannate or ammonium sulfate at a concentration of about 0.05 to 1.0% by weight, an enzyme at a concentration of about 0.02 to about 0.2% by weight and a flavor ingredient at a concentration of about 0.3 to about 1.0% by weight.

In the preparation of a oral rinse, an enzyme premix comprised of cetyl pyridinium chloride, reducing agent, water, humectant and enzyme is dispersed in a mixture of mouthwash ingredients, for example, alcohol, humectants, surfactants, and flavor are then added and mixed. The ingredients are then mixed under vacuum for about 15-30 minutes. The resulting oral rinse product is then packaged.

A rinse is an advantageous vehicle for delivering actives to the oral cavity, due to its ability to get into hard-to-reach areas of the mouth, such as the interproximal regions and the crevices of the tongue. The challenge in incorporating enzymes into a rinse is maintaining enzymatic stability and activity at water levels above 50%, conventionally not suitable for enzyme containing compositions. In the present invention, the stability of enzyme activity is found to be acceptable and is optimized unexpectedly when the water content of the rinse is maintained above 45% by weight of a mixture thereof, and preferably about 50 to about 85% by weight enzyme activity as an antiplaque agent is found to increase.

1. A dentifrice composition comprising a zinc-containing salt and carboxypeptidase, wherein said zinc-containing salt is not sequestered, and wherein the zinc is present in the amount of 0.3-0.6% by weight.

2. (canceled)

3. The composition according to claim 1, wherein the carboxypeptidase is present in the amount of 0.5% by weight.

3. The composition according to any of claim 1, wherein the ratio of zinc to carboxypeptidase is about 1:1.

4. The composition according to claim 3, wherein the dentifrice is a toothpaste.

5. The composition according to claim 3, wherein the dentifrice is a mouthwash.

6. The composition according to claim 3, wherein the dentifrice is a tooth powder.

7. The composition according to claim 1, wherein the zinc-containing salt is zinc citrate.

8. The composition according to any of claim 1, wherein the zinc-containing salt is zinc nitrate.

9. The composition according to claim 1, wherein the zinc-containing salt is zinc chloride.

10. A method of treating oral malodor comprising: administering a dentifrice composition according to claim 1.

11. A method of delivering anti-plaque benefits comprising: administering a dentifrice according to claim 1.

12. The method according to claim 10, wherein the dentifrice is a toothpaste.

13. The method according to claim 10, wherein the dentifrice is a mouthwash.

14. The method according to claim 10, wherein the dentifrice is a tooth powder.

15. A composition according claim 1, for use in a method of treating oral malador.

16. A composition according to claim 1, for use in a method of delivering antiplaque benefits.
